(19) United States
(12) Patent Application Publication
(10) Pub. No.: US 2015/0250908 A1
(43) Pub. Date: Sep. 10, 2015

(54) Intigrated Vehicle Hand Sanitizer

(71) Applicant: Christopher Arnold Maupin, Melbourne, FL (US)

(72) Inventor: Christopher Arnold Maupin, Melbourne, FL (US)

(21) Appl. No.: 14/202,036

(22) Filed: Mar. 10, 2014

(51) Int. Cl. A61L 2/18; A61L 2/24; B05B 12/12; B05B 11/00; B05B 12/00

(52) U.S. Cl. CPC A61L 2/18; B05B 11/3052; B05B 11/0089; B05B 12/004; B05B 12/122; A61L 2/24

(57) ABSTRACT

This invention relates to a hands free sanitizing system for use by drivers and passengers in an automobile other public conveyance. The mechanism comprises:
A reservoir to hold chemical—plastic or rubber;
A pump to transfer chemical;
An electrical harness to power pump;
A switch wire to trigger pump to turn on (sensory activated and/or manual);
Plastic or rubber insulated tubing to carry product (chemical) to one or more sprayer heads;
One or more sprayer head assemblies comprising a sprayer, manual button, sensor, light, and on/off switch;
Plastic couplings and fittings to attach tubing from reservoir to sprayer heads; and
One or more mounting brackets for the reservoir.

### FIELD OF THE INVENTION

The present invention relates to hands free, automatic, sensory or mechanically operated Hand Sanitation system to be provided in a private automobile or in a public conveyance, such as a bus, train or airplane. The integration of the system into the automobile or conveyance offers a hands free sanitation every time a driver and/or one or more passengers enter the car. The hand sanitation is available on demand and is effectuated by an electric sensor, by mechanical, or by any other means known in the art. The reservoir holds enough chemical for multi use. The system can be re-serviced by the vehicle owner, at a dealership, or at a service station. A fill port will be accessible either in the cab of the vehicle, or in the engine or storage compartments.

### BACKGROUND OF THE INVENTION

It is a known fact that the transmission of microbiological pathogens from the hands to other parts of the body or to people is a means of communicating diseases. Some potential pathogens are E coli, Salmonella, Shigella, Licteria, Staph, Auteus and the Flu Virus. The amount of germs on our hands when we enter our vehicles is astounding. The potential for being contaminated by dirty hands after leaving a store or entering a personal vehicle or public conveyance goes up tremendously, without the availability of some kind of hand sanitizer in the vehicle or conveyance. Typical washing of hands prior to leaving a restroom is inadequate because of the number of public surfaces people come into contact with on a constant basis. Having the extra protection of hand sanitation is a more effective way to kill the germs that may have been picked up prior to or upon entering a car or other conveyance.

With the Integrated Vehicle Hand Sanitizer in place the only thing a passenger's hands need touch is the outside of the vehicle. Once inside the vehicle or conveyance, the user glides his hands under a sensor to activate the Hand Sanitizer and start killing germs immediately. The chemical is then simply rubbed around on the hands, top and bottom, to kill those invisible germs that we all carry and cannot see. Typical hand sanitizing takes only a few seconds.

It is that initial hands free sanitation a user gets when a passenger enters a vehicle that will be the most effective in killing germs and bacteria. The fewer things that are touched when a person enters a conveyance, the less cross contamination will occur.

People in private automobiles or public conveyances are similar to a child in a Daycare Center, who will touch everything in their sight and then they put their hands to their mouths, nose, eyes and face. The cross contamination that ensues results in sickness and infection form germs and bacteria that enter the body through contact. This same process happens when we touch items in our car after leaving a store before we hand sanitize. The Integrated Vehicle Hand Sanitizer is the answer to keep individuals and the public healthier and safer by killing those germs and bacteria that we pick up on our hands in everything we touch.

### OBJECTS OF THE INVENTION

An object of the present invention is to provide an improved hands free way to sanitize the hands by dispensing an adequate amount of hand sanitizer to the users hand to manipulate around the hands for hands free sanitation.

Another object of the invention is to offer any time hand sanitation to the user because it is integrated into the car system. The system will have easy access to re-service system by the user, or at a Dealership or service station.

A further object of this invention is to encourage hand sanitation before and after every trip in a private automobile or public conveyance to help prevent the spread of disease, transient pathogens and the flu virus.

Additional objects of present invention are to get the consumer in the habit of sanitizing hands in between stops to cleanse hands and stop the spread of germs, and to facilitate the establishment of routine hand sanitizing amongst the public and improve public health.

### SUMMARY OF THE INVENTION

The integrated vehicle hand sanitizer uses an electric pump to dispense sanitizer chemical to the sprayer ports and onto the surface of the user's hands. The user will rub hands together and spread sanitizer around to kill germs. The Integrated Vehicle Hand Sanitizer comprises a container which houses chemical, a small pump to move chemical, tubing to transport chemical to conveniently located jets or ports throughout the passenger compartments of a personal vehicle or public conveyance, such as a taxi, bus, train or airplane. The ports will be operated either by a button or sensor light to activate pump, or by any other pumping means known to those having ordinary skill in the art.

The system will be integrated into the vehicle or public conveyance for easy access to service or refill system. The hands free system will give the vehicle driver and passengers the ability to sanitize hands without touching anything after people go to stores, open doors, touch shopping baskets, pump gas, handle money, shake hands or otherwise pick up germs and bacteria.

In one aspect of the present invention, the system uses a reservoir to house the chemical with a mini pump or pumping system known in the art, so that on demand the chemical can be dispersed from the sprayer port. The system uses a Hands Free sensor such as any known to those skilled in the art to dispense the chemical to the user. The system also has a dispenser button for optional discharge delivery. The sprayer device is provided with a colored light indicator system, to notify the user when the sanitizer is discharging and when the reservoir is empty, or approaching empty. For example, a red light could notify the user that the reservoir is empty. A green light could be used to indicate that the chemical is discharging. The system will also comprise an on and off switch at the sprayer port terminal to deactivate the system.

The system also has tubing that is connected to the reservoir through a mini pump which carries the chemical to the sprayer ports. The tubing can be made of any material known to the art, and may be insulated for extreme cold or extreme hot conditions.

Each jet port or nozzle will have an activation sensor that will send a signal to the mini pump to dispense for 2-3 seconds the right amount of chemical to the user to spread around user's hands. The amount of sanitizer dispensed and/or the length of time necessary to dispense the sanitizer is an amount of sanitizer/time dispensed effective to sanitize the hands of a user.

A button or other physical means can be installed for the user for override operation to activate the mini pump also used for priming the pump. The system would comprise a means to dispense a predetermined quantity of chemical onto the user's hands, such as a metering pump or a metering valve.

Additionally, the system may further include a means for indicating the operating status of the apparatus through the dashboard lights, similar to low fluid or system check lights.

A harness for the system wires may be provided. The wires with or without a harness for same may be connected to the system pump or to the reservoir with the system pump inside. The wire harness will either be connected through the fuse box or hard wired to the battery. The harness will include the power wire, ground wire, sensor activation wires and all wires needed to operate the system properly. The system can be powered as soon as a door opens in a car for quick and easy hands free access.

The system may be adapted with one or more ports to install at various stations in a personal vehicle or public conveyance. For example, one or two ports may be installed in the dashboard of a personal automobile. Multiple ports may be installed down the sides of a public transportation vehicle such as a bus or subway train. Individual ports may be installed in the overhead bins or armrests of airplanes. Obviously, multiple dispensation stations or ports might call for multiple reservoirs placed throughout the conveyance. Any and all other permutations of the applicant's integrated vehicle hand sanitizing system are considered by the inventor as being part of the present invention.

### BRIEF DESCRIPTION OF THE DRAWINGS

While the specification concludes with claims which particularly point out and distinctly claim the present invention, it is believed that the present invention will be better understood from the following description of the preferred embodiments, taken in conjunction with the accompanying drawings in which like reference numerals identify identical elements and wherein:

FIG. 1. Is the actual mechanical invention showing the progress of the system.

FIG. 2. Are the components that make up the Integrated Dashboard Hand Sanitizer System.

### DETAILED DESCRIPTION OF THE INVENTION

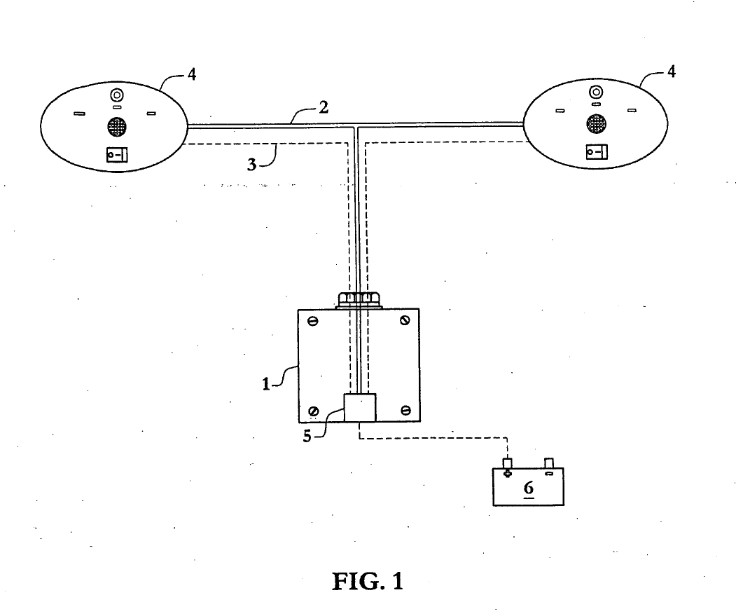

In a preferred embodiment shown in FIG. 1, the present invention provides an Integrated Hand Sanitation System that can be installed in the automobile. In FIG. 1 the main components of the Integrated Dashboard Hand Sanitizer are:
A. Reservoir to house chemicals
B. Integrated tubing to transfer product
C. Sensor wire to tell the mini pump to activate the right amount of chemical.
D. Dashboard sprayer module, encompassing the components to activate disbursement of chemical.
E. Mini pump the component to actually pump chemical to dashboard sprayer module.

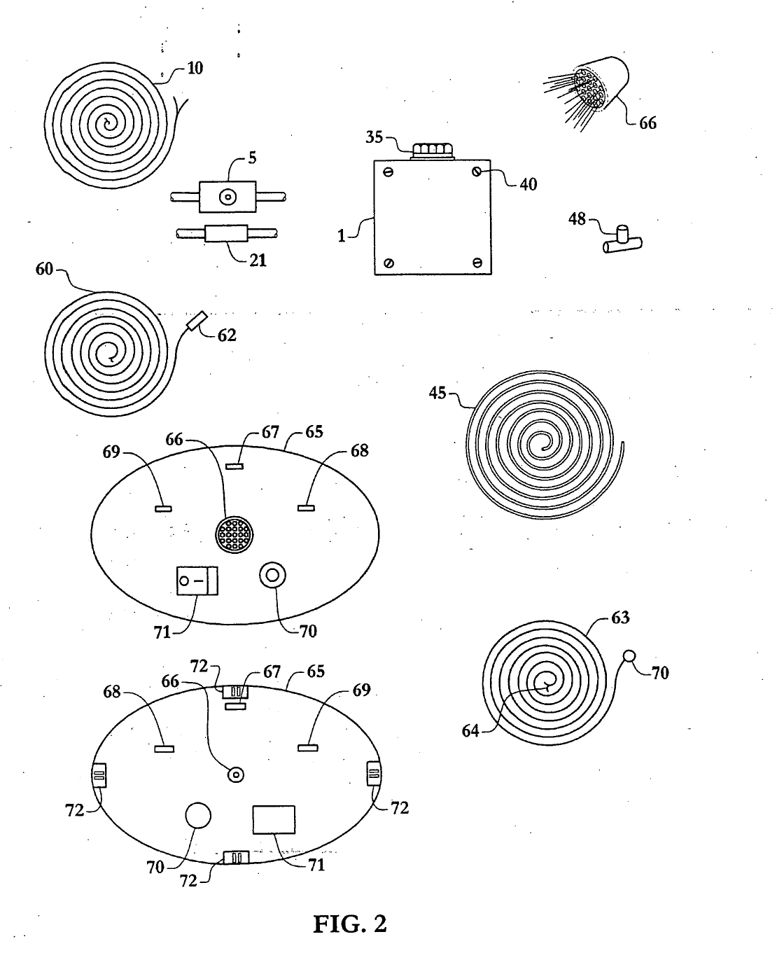

In FIG. 2 This picture shows all components that are used in the in dash hand sanitizer system.

#10 is the pump power wire. This wire is used to energize the mini pump. The power source will come through the fuse box and have its own fuse.
#20 is the mini pump, it is used to transfer chemical from the reservoir to the sprayer heads.
#30 is the reservoir unit used to store chemical. The reservoir can be mounted on the firewall in the engine compartment or under the dashboard inside the care.
#35 is the cap of the reservoir similar to the cap on a windshield washer reservoir, located conveniently in the vehicle for easy access to reservoir.
#40 is the mounting screws adaptable for each type of vehicle.
#45 is the plastic tubing used to transfer chemical to sprayer heads from the mini pump. The tubing will attach to the mini pump and travel out to the sprayer heads mounted on the dash board.
#48 is directional plastic couplings used to attach lengths of tubing to desired locations. The couplings will be different angles used to apply to specific locations of the sprayer heads, depending on vehicle dashboards. The tubing will tightly slip over nipple of sprayer head and nipple of mini pump.
#50 is the sprayer jet. The jet is similar to the jet used on window washer on car windshield.

Known in the arts, the jet will be metered thru the mini pump for a predetermined amount of chemical to be discharged to user's hands for application. There can also be an inline metering valve #21 to be used to establish desired amount of chemical to user's hands.

#60 sensor wire harness will carry the signal thru to the pump #20 which will be activated by the photo sensor when the user passes his hand in front of the photo sensor #62. The photo sensor, which will be mounted next to the sprayer jet #50 so users hand will be in place when photo sensor is activated. There is a green light #69 next to the sprayer jet letting user know that the system is working. At this time the desired amount of chemical will discharge into user's hands. There is also a manual button #70 that will be next to the on/off switch #71 that can be used to prime pump and also override photo sensor. The manual button #71 is just another way the user can dispense product (chemical) to hands. It also can be used to prime mini pump. The on/off switch #71 is a slider type switch located at the bottom of the sprayer module unit to turn unit to turn on and off system. There is a red light #68 located opposite the green light #69 to let user know what configuration system is in. Red light #68 lets user know system is turned off. #71 the on/off switch will be wired directly to the power source through the wiring harness (pump power wire) #10 to energize the mini pump. The manual button #70 next to the on/off switch #71 will have a wire #63 that will run to the mini pump #20 through wire harness #10 to deliver signal to activate pump #20, the end of the wire #63 will have wire connectors to connect to the power source #64 wire connectors to complete manual button connection. When the user activates photo sensor #67 the dispense cycle will begin, the mini pump #20 will activate for 2 to 3 seconds to dispense chemical on users hand. When cycle is complete the user will have to pass photo sensor again to get another 2-3 second cycle. The user can tap the manual button with outside of index finger to activate mini pump manually #20. The mini pump #20 will cycle through as if photo sensor was activated #67, allowing 2-3 seconds of chemical to disperse. The location of the dashboard sprayer module #65 will be up to the manufacturer, someplace on the dashboard for easy access. The module itself
#65 will hold all components that operate hand sanitation system, including #66, sprayer jet, to dispense chemical #67 photo sensor to activate system #71 on/off switch to control system, #70, manual button to override sensor or another way to get chemical also for priming mini pump. The green light #69 letting user know product is dispensing, red light #68 lets user know system is turned off.

The backside of the Dashboard Sprayer module will have 4 plastic snaps #72 that will snap into dashboard at desired location. The sprayer module can be round, square or rectangle, whichever shape accommodates user and vehicle make. The dashboard sprayer module will have pre cut opening for all components #69 green light, #67 photo sensor, #66 sprayer head, #68 red light, #70 manual button, #71 on/off switch to all be snapped into place. The dashboard sprayer module #65 will mount flush into the dashboard for easy accessibility and sleek design to flow with the car's interior.

1. A hands free sanitizing system for use by drivers and passengers in an automobile or other public conveyance which comprises:
a reservoir to help sanitizer
a pumping means to transfer sanitizer
a power source for said pumping means
at least one sprayer head assembly
tubing to deliver sanitizer to said sprayer head assemblies; and actuating means to initiate delivery of said sanitizer

2. A system according to claim 1, where in the reservoir is plastic or rubber.

3. A system according to claim 1, where in the power source is electrical power source.

4. A system according to claim 1, where in the sprayer head assembles comprise a sprayer, a sensor, an indicator light, and a on/off switch.

5. A System according to claim 1, where in the actuating means it is a manual switch.

6. A system according to claim 1, wherein the actuating means comprises a motion sensor.

7. A system according to claim 1, wherein the sprayer head assemble can be made of plastic with snaps to mount in conveyance. Either snapped in or screwed into place.

8. A system according to claim 1, where in the tubing can be rubber, plastic, copper or any type of material that can carry product to user ports.

9. A system according to claim 1, wherein tubing is attached to conveyance by snaps, straps, clamps, ties, rubber band or any kind of connecting material.

10. A system according to claim 1, wherein the system pump is a standard mini pump used in all types of systems to extract liquid into the pump and out to the sprayer ports. The pump can be submerged into the reservoir or outside reservoirs to transport fluid.

11. A system according to claim 1, where in the fluid in the reservoir can be a gel type liquid. The fluid can also be a water base alcohol type to allow for finer mist spray.

12. A system according to claim 1 wherein the plastic or rubber reservoir can be 1 gallon or smaller to house enough chemical to allow for multiple uses.

13. A system according to claim 1, wherein the reservoir can be mounted with a screw, nuts, bolts, plastic snaps, straps, rubber, plastic, wire straps.

14. A system according to claim 1, where in the reservoir will have a screw on or a pop off lid for easy re service and excess to pump is housed inside reservoirs.

15. A system according to claim 1, where in the regulator can be plastic, rubber or any material that can be used to regulate the amount of chemical to the user. The regulator will be inline the tubing system either before or after the mini pump.

16. A system according to claim 1 wherein the sprayer modular head can be a shower type of head, mist, jet, flush, or any type head to deliver product to user.

17. A system according to claim 1, where in the power supply to system will come through fuse box with separate fuse from conveyance battery system to keep power to the sanitizer system. Power can also come straight to system from the battery with its own inline fuse in wire harness.

18. A system according to claim 1, wherein the wire harness is encumbered in plastic tubing, rubber or any type of tubing that is safe for wiring. It can be insulated for fire safety and mounted to the conveyance with screw clamps, plastic strap ties, rubber ties, or any material that can work efficiently with proper performance.

19. A system according to claim 1, wherein the lighting indicator on the sprayer head assembly will be led type to allow for adequate lighting to inform the user of system readiness. The wiring behind the sprayer assembly will be attached to the assembly by wire connector.

20. A system according to claim 1, wherein the on/off switch on sprayer assembly can be plastic, aluminum, metal or any type of switch to control sprayer assembly. The manual over ride switch will be a button type with spring behind activator.
